(19) United States
(12) Patent Application Publication
(10) Pub. No.: US 2025/0212896 A1
(43) Pub. Date: Jul. 3, 2025

(54) PROCESS FOR THE PREPARATION OF A HYDROLYZED EXTRACT OF NON-ANIMAL ORIGIN

(71) Applicant: SICIT GROUP S.P.A., CHIAMPO (VI) (IT)

(72) Inventors: Manuel ASNICAR, CHIAMPO (VI) (IT); Alberto CATTAZZO, COLOGNOLA AI COLLI (VR) (IT); Massimo Costantino NERESINI, CHIAMPO (VI) (IT); Alberto RUMIGNANI, VALDAGNO (VI) (IT)

(21) Appl. No.: 18/850,406

(22) PCT Filed: Mar. 30, 2023
(86) PCT No.: PCT/EP2023/058299

§ 371 Date: Sep. 24, 2024

(30) Foreign Application Priority Data
Apr. 1, 2022 (IT) 102022000006539

(51) Int. Cl. A01N 65/03; A01N 63/20; A01N 63/32; A01N 65/08; A01N 65/20; A01P 21/00; C12P 7/24; C12P 13/04; C12P 33/00

(52) U.S. Cl. CPC A01N 65/03; A01N 63/20; A01N 63/32; A01N 65/08; A01N 65/20; A01P 21/00; C12P 7/24; C12P 13/04; C12P 33/00

(57) ABSTRACT

The present invention relates to a process to obtain a hydrolyzate from a biomass of non-animal origin. The invention further relates to the hydrolyzate of non-animal origin obtained by said process and the use thereof as a plant biostimulator.

### TECHNICAL FIELD OF THE INVENTION

The present invention relates to a process to obtain a hydrolyzate from a biomass of non-animal origin, for example from plants or plant parts, seed flours, algae, yeasts, etc., the hydrolyzate of non-animal origin obtained by said process and the use thereof as a plant biostimulator.

### BACKGROUND ART

Hydrolyzates are an important group of plant biostimulators mainly containing a mixture of oligosaccharides, polyphenols, peptides and amino acids, phytohormones, etc. which have demonstrated significant potential in improving productivity, nutritional quality, and tolerance to abiotic stressors of plants in various agricultural crops. The use of hydrolyzates of plant origin as biostimulators constitutes a valid alternative to the application of chemical fertilizers having negative effects for people's health and the environment.

It was now found out that a process providing for the use of the high-pressure homogenization technique with specific process conditions before the enzymatic hydrolysis allows to obtain hydrolyzates of non-animal origin with high hydrolysis degree able to stimulate the plant development providing one or more nutrients such as oligosaccharides, polyphenols, amino acids, peptides, and nitrogen in various forms. Such nutrients are absorbable by the plant directly through the leaf system or from the ground through the root system.

The High-Pressure Homogenization (HPH) is a mechanical method used for the rupture of the cell wall of cells of various origins, for example microalgae (Spiden Bioresource Technology 140 (2013) 165-171; Safi et al. Algal Research 3 (2014) 55-60); (Barba et al. Food Eng Rev (2015) 7:45-62) in order to release the cellular content.

The use of the high-pressure homogenization technique with particular process conditions instead of the homogenization with a rotor-stator system before the enzymatic treatment allows to obtain hydrolyzates of non-animal origin with better characteristics, such as greater recover of amino acids and high extraction yields and high nitrogen content.

### BRIEF DESCRIPTION OF THE FIGURES

FIG. 1 schematically shows a process according to the invention.

FIG. 2 shows a process according to the invention starting from nettle leaves.

FIG. 3 shows a process according to the invention starting from defatted soy flour.

### SUMMARY OF THE INVENTION

Object of the present invention is a process to obtain hydrolyzates of non-animal origin comprising the steps of:
i) subjecting a biomass to water infusion with a dry biomass/water ratio comprised between 1:5 and 1:20, at a temperature between 20° C. and 45° C., separating a liquid phase from the solid biomass;
ii) subjecting the solid biomass obtained after step i) of non-animal origin to two or more high-pressure homogenization cycles wherein the homogenization cycles are performed at pressures from 200 bar (20000 kPa) to 2000 bar (200000 kPa);
iii) subjecting the thus obtained homogenized to one or more enzymatic hydrolysis cycles wherein in each cycle one enzyme or more enzymes are used, selected from polysaccharide-degrading enzymes, endo-proteolytic enzymes, or exo-proteolytic enzymes;
iv) performing enzymatic inactivation; and
v) recovering the hydrolyzate of non-animal origin and rejoining with the liquid phase obtained in step i).

A further object of the present invention are hydrolyzates of non-animal origin obtained by the process of the invention and the use thereof as plant biostimulators.

### DETAILED DESCRIPTION OF THE INVENTION

Object of the present invention is a process to obtain hydrolyzates of non-animal origin comprising the steps of:
i) subjecting a biomass to water infusion with a dry biomass/water ratio comprised between 1:5 and 1:20, at a temperature between 20° C. and 45° C., separating a liquid phase from the solid biomass;
ii) subjecting the solid biomass obtained after step i) of non-animal origin to two or more high-pressure homogenization cycles wherein the homogenization cycles are performed at pressures from 200 bar (20000 kPa) to 2000 bar (200000 kPa);
iii) subjecting the thus obtained homogenized to one or more enzymatic hydrolysis cycles wherein in each cycle one enzyme or more enzymes are used, selected from polysaccharide-degrading enzymes, endo-proteolytic enzymes, or exo-proteolytic enzymes;
iv) performing enzymatic inactivation; and
v) recovering the hydrolyzate of non-animal origin and rejoining with the liquid phase obtained in step i).

The term “biomass” generally means the biodegradable fraction of products, waste, by-products, and residues of biological origin deriving from agriculture (comprising plant substances), forestry and related industries and also from the food industry. The biomass may be of plant or microbial origin.

The biomass of non-animal origin used in the process of the present invention, preferably of plant or microbial origin, may be consisted of:
plants or plant parts such as leaves, branches, roots, or seeds. Preferred plants are moringa spp., preferably Moringa oleifera Lam., urtica spp., nabo (Brassica rapa spp.), cassava (or manioc or Manihot esculenta), leucaena (Leucaena leucocephala), lupine (Lupimis albus) and cajanus (or pigeon pea or Cajanus cajan), etc.;
seed flours such as soy, sunflower, moringa, hemp and rapeseed flours, etc.;
microalgae selected from chlorella, such as Chlorella vulgaris spp., spirulina spp., etc.;
yeasts such as brewer's yeast, etc.

The term “flour” means the seed cake flour remaining after the process of extraction of the seed oil.

The biomass of non-animal origin may be in fresh or dried form.

The biomass in dried form is preferably available in powder form. If not in powder form, it can be ground for example with a blade mill. The powder particle size is preferably ≤0.5 mm.

The fresh biomass may be ground before being subjected to the subsequent process steps. The powder particle size is preferably ≤0.5 mm. Microalgae may be used as such.

Before homogenization, the ground or powdered biomass is diluted to a dry substance (DS): water ratio from 1:10 to 1:25, preferably 1:20.

In the case of algae coming from a reactor, or photobioreactor cultivation, the biomass is concentrated, preferably by micro/ultra-filtration or centrifugation, to a dry substance (DS): water ratio from 1:10 to 1:25, preferably equal to 1:20.

Before the homogenization step, the biomass is subjected to water infusion with a dry biomass/water ratio comprised between 1:5-1:20, at a temperature between 20° C. and 45° C., preferably at room temperature, for example about 25° C. (cold extraction of hydrophilic molecules). The infusion may occur in reactors under stirring or not. Such operation allows to extract thermosensitive water-soluble molecules, preserving them from degradation due to the higher temperatures used in the homogenization and hydrolysis steps. After the infusion, the liquid is separated by centrifugation, possibly concentrated with techniques operating at a temperature not higher than 45° C., for example by nanofiltration and reverse osmosis; or direct osmosis; or a combination of the preceding techniques, and rejoined to the hydrolyzed biomass in the process final steps, when the temperatures are <45° C. The solid biomass, coming from centrifugation is re-dispersed in water at a ratio dry substance (DS): water from 1:10 to 1:25, preferably 1:20 and intended to the subsequent homogenization step.

In addition to the extraction of hydrophilic molecules, the biomass may be subjected to the cold extraction of lipophilic molecules, for example by extraction with supercritical CO₂ possibly adjuvated from co-solvents such as ethanol and/or water. The lipophilic extract is separated and intended for other uses such as in formulations for foliar application, in seed treatment processes, as additive in biostimulator formulations, etc.

### High-Pressure Homogenization

The high-pressure homogenization cycles are preferably two, three or four and May be performed at the same pressure or at increasing pressures based on the biomass.

The homogenization pressure is selected in the range between 200 bar (20000 kPa) and 2000 bar (200000 kPa), such as 200 bar (20000 kPa), 400 bar (40000 kPa), 800 bar (80000 kPa), 1000 bar (100000 kPa), 1200 bar (120000 kPa) or 1500 bar (150000 kPa) or any intermediate value between these.

Generally, “hard” raw materials, such as Moringa branches, have to be processed initially at low pressures, in order to avoid the stopping of the homogenizer. On the contrary, “soft” raw materials, such as Spirulina, may be processed directly at high pressures.

### Enzymatic Hydrolysis

The enzymatic hydrolysis comprises the following steps:
a) possible pH adjustment between 4.5 and 6.0, preferably 5.5;
b) possible adding of an enzymatic inoculum of polysaccharide-degrading enzymes e.g., cellulases and/or hemicellulases and/or glycosidases and/or pectinases and/or chitinases and/or chitosanases etc., and execution of an enzymatic infusion for degrading polysaccharides at a temperature comprised between 36 and 73° C., preferably 60° C.;
c) pH adjustment between 6.0 and 8.0, preferably between 6.0 and 7.0 or between 7.0 and 8.0, more preferably between 6.5 or 7.4;
d) adding of at least one endo-proteolytic enzyme and execution of an endo-proteolytic enzymatic infusion at a temperature comprised between 4° and 50° C., preferably 45° C. or at a temperature comprised between 6° and 75° C., preferably 68° C.;
e) pH adjustment between 6.0 and 7.0, preferably 6.5;
f) adding of at least one exo-proteolytic enzyme and execution of an exo-proteolytic enzymatic infusion at a temperature comprised between 4° and 50° C., preferably 45° C.;
g) centrifugation and/or filtration to separate the liquid phase from the solid phase;
h) concentration of the liquid phase, preferably by nanofiltration and reverse osmosis; or under vacuum concentration; or direct osmosis; or combinations thereof.

The solid residue obtained in step g) may be dried and used in solid formulations or composted for the production of soil conditioners.

The enzymatic hydrolysis occurs by adding an enzymatic inoculum of polysaccharide-degrading and/or endo-proteolytic and/or exoproteolytic enzyme and enzymatic infusion. The inoculum amount, pH, temperature, and infusion time may be selected based on the enzyme/enzymes used and substrate to be hydrolyzed.

The inoculum amount may vary, depending on the used enzyme and biomass type, from 0.01% w/w to 2.0% w/w referred to the dry weight of the biomass of non-animal origin under processing.

The polysaccharide-degrading enzymes hydrolyze the polysaccharide components of the cell wall. Depending on the polysaccharide composition the more suitable enzyme or enzymes for the hydrolytic action such as cellulases and/or hemicellulases and/or glycosidases (for example 1,4-β-D-glycosidase) and/or pectinases and/or chitinases and/or chitosanases etc. may be used. Examples of polysaccharide-degrading enzymes are those of the line Cellusoft® of Novozymes.

The polysaccharide-degrading enzymes are generally used at a pH between 5.5 and 9.0 and a temperature between 3° and 70° C.

Endo-proteolytic enzymes (endo-peptidases) are selected from alcalases, serin proteases. Examples of endo-proteolytic enzymes are Alcalase® of Novozymes, Prolyve NP®.

The endo-proteolytic enzymes are generally used at a pH between 5.5 and 9.0 and a temperature between 4° and 70° C.

Exo-proteolytic enzymes (exo-peptidases) are selected from aspartyl proteases.

An example of exo-proteolytic enzyme is Prolyve NP®.

The enzymatic pool derived from A. oryzae (Aspergillus oryzae), for example Prolyve NP®, consisting of a mixture of proteases with endo- and exo-peptidase activity, may be used, preferably comprising an aspartyl protease, also characterized by amylase and hemicellulose activity, having an endo-proteolytic activity at a pH comprised between 7.0 and 8.0, preferably 7.4, and exo-proteolytic activity at a pH comprised between 6.0 and 7.0, preferably 6.5.

### Enzymatic Inactivation and Hydrolyzate Recovery

i) The enzymatic inactivation may occur by heating at a temperature between 3° and 50° C. and/or acidification at a pH between 3.8 and 5.0, and/or subjecting the mixture at a pressure between 20 mbar (2000 Pa) and 200 mbar (20000 Pa) for a period of time between 15 and 30 minutes.
   The solid residue obtained in step g) may be dried and used in solid formulations or composted for the production of soil conditioners.

The choice of these parameters depends on the used enzyme/enzymes.

Before the enzymatic inactivation, the liquid phase is separated from the solid by centrifugation and/or filtration and concentrated for example by nanofiltration and reverse osmosis; or under vacuum concentration; or direct osmosis; or a combination of the preceding techniques.

The thus obtained product may be mixed with the aqueous extract obtained before homogenization (cold extraction of hydrophilic molecules) which was optionally concentrated separately with techniques operating at a temperature not higher than 45° C., for example by nanofiltration and reverse osmosis; or direct osmosis; or a combination of the preceding techniques. Optionally additives such as microelements, preservatives, lipophilic components (see extraction with supercritical CO₂) and emulsifiers may be added.

The thus obtained concentrated product is preferably left to settle for a few days, preferably 1-2 weeks (maturation), and then filtered and/or centrifugated to give the hydrolyzate of non-animal origin. The maturation step increases the final product physical stability eliminating the poorly soluble substances. The solid residue obtained in the separation step at the end of maturation may be dried and used in solid formulations or composted for the production of soil conditioners.

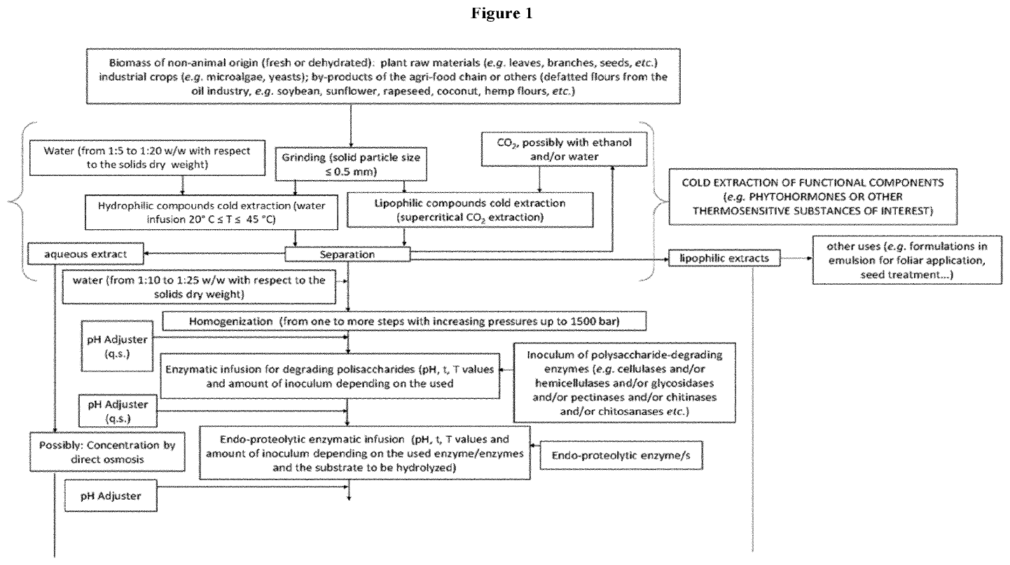

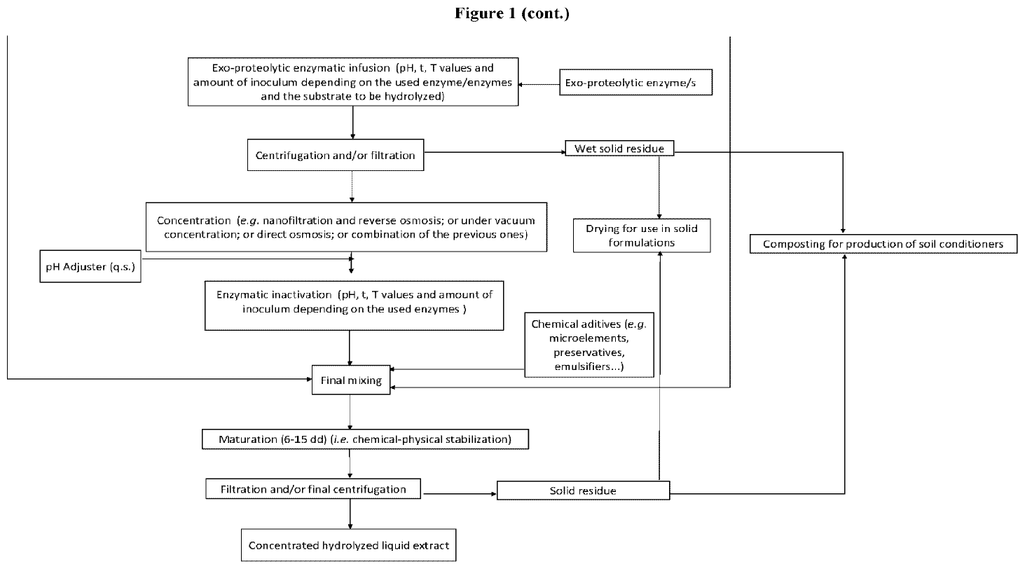

FIG. 1 summarizes schematically a process according to the invention.

Object of the present invention are also the hydrolyzates of non-animal origin obtained by the process of the invention and the use thereof as plant biostimulators.

The term “biostimulator” according to the present invention means a substance which, when applied to plants, is able to ameliorate the nutritive efficiency, the abiotic stress tolerance and/or the qualitative characteristics of crops, independently of its nutrient content.

The following examples further illustrate the invention.

### EXAMPLES

### Definitions and Analytical Methods

DS is the dry substance.

The dry substance is determined by oven drying at the temperature of 105° C. until a constant weight is reached.

Nₜₒₜ is the total nitrogen content.

Cₜₒₜ is the total carbon content.

The method for determining the total nitrogen and total carbon contents is based on the complete combustion of the sample in the presence of oxygen. To obtain a complete oxidation, the sample is carried by a helium flow through a column filled with a catalyst, chromium oxide (Cr₂O₃). The nitrogen oxides formed in the column are then reduced in a copper column retaining oxygen also. The thus formed substances, N₂, CO₂ and H₂O, pass through a filter retaining water. CO₂ and N₂ are separated by column chromatography and the combustion product analysis is performed by gas chromatography (elementary analysis). The total nitrogen and the carbon in the extracts were reported as dry substance percentage.

The extraction yield Ye DS was calculated on the initial dry substance in the sample as follows:

$${Y}_{e / DS} = \frac{me}{mDS} \cdot 100$$

wherein mₑ(g) is the dry substance in the extract and m_{DS} is the solid substance in the initial sample (g).

Nᵣ is the nitrogen recovery (%) expressed as [ratio between the nitrogen measured in the extract (Nₜₒₜ, extract) and the total nitrogen in the initial sample (Nₛₐₘₚₗₑ)]×100.

The free amino acid content is the ratio between free and total amino acids in the hydrolyzate.

The total and free amino acids were determined by a HPLC fluorometric analysis. Specifically, the total amino acids were analyzed after acid hydrolysis, which was performed using 6N HCl in the presence of 1 g L-1 of phenol. The free amino acids were determined directly, without the hydrolysis step. The liquid chromatography (Agilent1-STRU043) was performed after the column derivatization with 100 mg of o-phthaldialdehyde (OPA), which was dissolved in 2 mL of methanol, in the presence of 0.08 mL of 3-mercaptopropionic acid for the derivatization of the primary amino acids. Then, 77.5 mg of 9-fluorenilmethyl chloroformate (FMOC), dissolved in 20 mL of acetone, were used as reagents for the derivatization and determination of the secondary amino acids. The analysis was performed by a fluorescence detector (FLD Spectra Infinity 1260 Agilent). For tryptophan determination, before performing the analysis, basic hydrolysis with 4.2N NaOH was performed. As above described, the analysis was performed by liquid chromatography and the used instrument (FLD Spectra Infinity 1260 Agilent) was equipped with a fluorescence detector. The column derivatization was performed with OPA in the presence of 3-mercaptopropionic acid before performing the analysis.

### EXAMPLE

### Experimental Procedure

The powdered raw material (Chlorella, Spirulina, Moringa (Branches), Moringa (Leaves), SCW Yeast-(brewer's yeast), EF Soy-extraction flour) is mixed with water according to the ratios set in Table 1.-step 1.

The thus prepared mixture is subjected to one or more homogenization cycles in a high-pressure homogenizer. Generally, the “hard” raw materials, such as Moringa branches, have to be processed initially at low pressures, in order to avoid the stopping of the homogenizer. On the contrary, “soft” raw materials, such as Spirulina, may be processed directly at high pressures.

The number of the homogenization cycles and the homogenization pressures are detailed below for each raw material.

Once homogenized, the suspension is reduced in volume, if required, as indicated in Table 1.-step 2, and the solid content % is determined with a thermobalance (110° C.).

The suspension is then subjected to enzymatic hydrolysis, enzymatic inactivation, and recovery in the conditions set in Table 1.-step 2.

The tests are performed in triplicate and the results are an average of the three tests performed.

TABLE 1

| | Step 1 | Step 2 | Step 3 Enzyme Inactivation | |
|---|---|---|---|---|
| Raw material | Homogenization | Enzymatic Hydrolysis | and Recovery | Example |
| Chlorella | High-pressure | Ratio 1:20 | 85° C., 15 min | 01 |
| | homogenizer | ALCALASE 0.75%, pH 9.0, 68° C., 60 min | Centrifugation and | |
| | Ratio 1:20 | PROLYVE NP Liquide 0.30%, pH 6.8, 45° | Supernatant Recovery | |
| | | C., 60 min | | |
| Spirulina | High-pressure | Ratio 1:20 | 50° C., pH 4.0, 15 min, | 02 |
| | homogenizer | CELLUSOFT 19500L 0.75% & PROLYVE NP | Centrifugation and | |
| | Ratio 1:20 | Liquide 0.30%, pH 6.8, 40° C., 60 min | Supernatant Recovery | |
| | | then 45° C., 60 min | | |
| Moringa | High-pressure | Ratio 1:10 | 85° C., 15 min | 03 |
| (Branches) | homogenizer | CELLUSOFT SUPREME 0.5%, pH 8.5, 45° C., | Centrifugation and | |
| | Ratio 1:20 | 60 min | Supernatant Recovery | |
| | | ALCALASE 0.5%, pH 7.0, 60° C., 60 min | | |
| | | PROLYVE NP Liquide 0.6%, pH 6.8, 45° C., | | |
| | | 60 min | | |
| Moringa | High-pressure | Ratio 1:10 | 50° C., pH 4.0, 15 min, | 04 |
| (Leaves) | homogenizer | CELLUSOFT 19500L 0.5%, pH 6.8, 40° C., | Centrifugation and | |
| | Ratio 1:20 | 60 min | Supernatant Recovery | |
| | | PROLYVE NP Liquide 0.2%, pH 6.8, 45° C., | | |
| | | 60 min | | |
| SCW Yeast | High-pressure | Ratio 1:10 | 85° C., 15 min | 05 |
| | homogenizer | ALCALASE 0.50%, pH 7.0, 60° C., 60 min | Centrifugation and | |
| | Ratio 1:20 | | Supernatant Recovery | |
| EF Soy | High-pressure | Ratio 1:20 | 85° C., 15 min | 06 |
| | homogenizer | CELLUSOFT SUPREME 0.125%, pH 6.0, | Centrifugation and | |
| | Ratio 1:20 | 60° C., 60 min | Supernatant Recovery | |
| | | ALCALASE 0.125%, pH 9.0, 68° C., 60 min | | |
| | | PROLYVE NP Liquide 0.125%, pH 6.8, 45° | | |
| | | C., 240 min | | |
| Nettle | High-pressure | Ratio 1:20 | 85° C., 15 min | 07 |
| | homogenizer | CELLUSOFT SUPREME 0.125%, pH 6.0, | Centrifugation and | |
| | Ratio 1:20 | 60° C., 60 min | Supernatant Recovery | |
| | | ALCALASE 0.125%, pH 9.0, 68° C., 60 min | | |
| | | PROLYVE NP Liquide 0.125%, pH 6.8, 45° | | |
| | | C., 240 min | | |

### Example 1-Chlorella

Homogenization-I. 400 bar, II. 800 bar, III. 1500 bar. After homogenization, a slow formation of a precipitate occurs.

The extract subjected to homogenization and subsequent enzymatic treatment appears as a green opalescent liquid.

### Example 2-Spirulina

Homogenization-I. 400 bar, II. 800 bar, III. 1500 bar. After homogenization, a slow formation of a precipitate occurs. During processing, Spirulina tends to form a lot of foam.

The extract subjected to homogenization and subsequent enzymatic treatment appears as a blue semi-transparent liquid.

### Example 3-Moringa (Branches)

Homogenization-I. 200 bar, II. 200 bar.

The extract subjected to homogenization and subsequent enzymatic treatment appears as a brown opalescent liquid.

### Example 4-Moringa (leaves)

Homogenization-I. 200 bar, II. 200 bar.

The extract subjected to homogenization and subsequent enzymatic treatment appears as a brown transparent liquid.

### Example 5-SCW Yeast

Homogenization-I. 500 bar, II. 1000 bar, III. 1500 bar.

The extract subjected to homogenization and subsequent enzymatic treatment appears as a yellow opalescent liquid.

### Example 6-EF Soy

Homogenization-I. 400 bar, II. 800 bar, III. 1200 bar.

The extract subjected to homogenization and subsequent enzymatic treatment appears as an off-white opalescent liquid.

### Example 7-Nettle

Homogenization-I. 200 bar, II. 400 bar, III. 800 bar.

The extract subjected to homogenization and subsequent enzymatic treatment appears as a dark green transparent liquid.

### Example 8-Comparative hydrolyzate

The data obtained with the homogenization step in a high-pressure homogenizer, (hereinafter “HPO”) were compared with the results obtained on the same raw materials, homogenized with a homogenizer with a rotor-stator system i.e. ULTRA-TURRAX® T-25 of IKA, hereinafter “UTX”.

The raw material was suspended in water at a ratio solids/water w/w of 1:20 and subjected to 1 minute homogenization at the highest speed (22500 rpm) and 1 minute rest, repeated 4 times.

Then, the samples were subjected to enzymatic hydrolysis, enzymatic inactivation, and recovery in the same conditions of the samples subjected to the high-pressure homogenization.

### Results

The results are set in the following Table 2.

TABLE 2

| Raw material | Treatment | N_{Tot}/DS | N_{Tot}/C_{Tot} | AA_{Tot}/DS | AA_{Free/Tot} | Ext Yield | AA Recovery | K⁺/SS |
|---|---|---|---|---|---|---|---|---|
| Chlorella | Raw Material | 8.91% | 19.30% | 53.00% | — | — | — | — |
| | UTX & Enzymes | 10.34% | 19.24% | 53.67% | 14.46% | 51.53% | 61.43% | 5.30% |
| | HPO & Enzymes | 15.17% | 25.82% | 77.30% | 17.05% | 42.54% | 64.50% | 5.96% |
| Spirulina | Raw Material | 11.18% | 23.37% | 60.00% | — | — | — | — |
| | UTX & Enzymes | 10.54% | 21.68% | 55.13% | 21.75% | 58.79% | 59.01% | 2.48% |
| | HPO & Enzymes | 11.10% | 19.35% | 64.38% | 26.29% | 53.21% | 60.69% | 3.08% |
| Moringa | Raw Material | 1.10% | 2.38% | 5.80% | — | — | — | — |
| (Branches) | UTX & Enzymes | 9.31% | 8.05% | 9.50% | 23.94% | 20.54% | 63.74% | 13.59% |
| | HPO & Enzymes | 5.06% | 9.65% | 16.48% | 37.75% | 27.18% | 81.19% | 8.85% |
| Moringa | Raw Material | 4.70% | 9.96% | 24.70% | — | — | — | — |
| (Leaves) | UTX & Enzymes | 4.65% | 9.03% | 20.42% | 32.45% | 30.29% | 33.31% | 3.37% |
| | HPO & Enzymes | 8.22% | 15.63% | 37.01% | 28.42% | 46.01% | 72.55% | 7.33% |
| SCW Yeast | Raw Material | 7.84% | — | 46.90% | — | — | — | — |
| | UTX & Enzymes | 9.58% | 18.14% | 54.81% | 13.45% | 52.49% | 59.20% | 3.59% |
| | HPO& Enzymes | 11.44% | 23.08% | 59.39% | 12.57% | 49.09% | 67.14% | 4.05% |
| EF Soy | Raw Material | 8.74% | 18.86% | 48.43% | — | — | — | — |
| | UTX & Enzymes | 10.65% | 20.40% | 66.79% | 0.47% | 53.30% | 72.36% | 6.00% |
| | HPO& Enzymes | 12.67% | 24.78% | 73.74% | 10.84% | 55.94% | 95.01% | 5.36% |
| Nettle | Raw Material | 3.77% | 9.34% | 16.74% | — | — | — | — |
| | UTX & Enzymes | 5.48% | 12.92% | 26.54% | 1.89% | 29.61% | 46.63% | 9.99% |
| | HPO & Enzymes | 8.66% | 17.70% | 26.05% | 23.93% | 32.16% | 54.17% | 11.66% |

### CONCLUSIONS

From data set in Table 2 it is clear how the high-pressure homogenizer results in a higher extraction yield of the components of interest listed in the Table 2, following a more effective access to the cellular content.

### Example 9

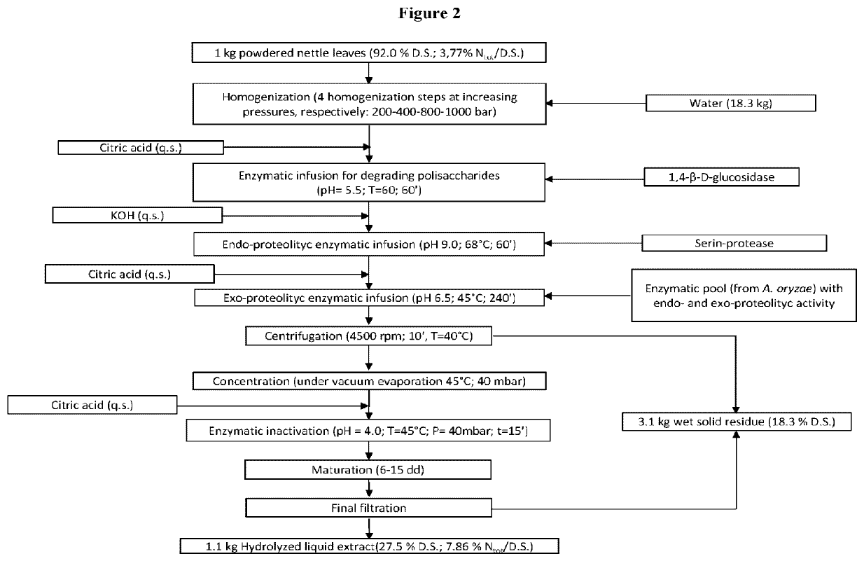

1 kg of nettle leaves were treated as described in the scheme of FIG. 2.

### Example 10

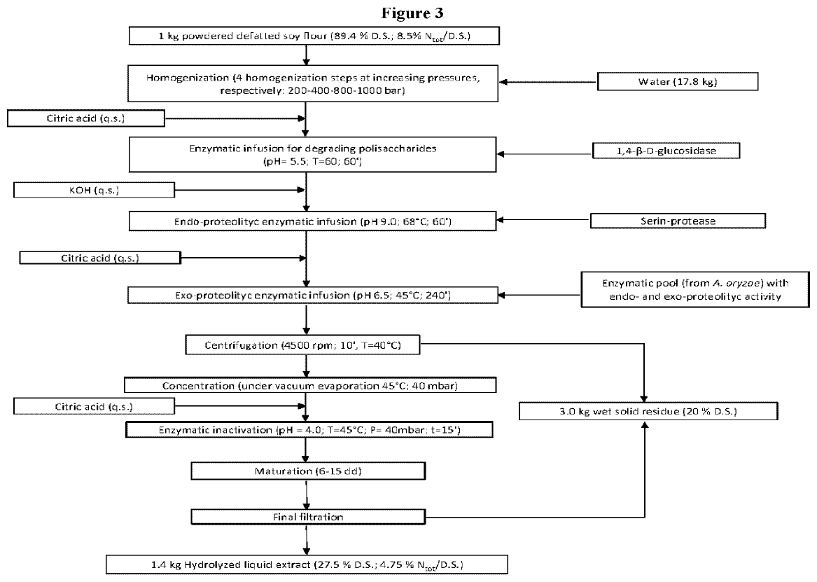

1 kg of defatted soy flour was treated as described in the scheme of FIG. 3.

### Example 11

10 kg of dried powder of moringa biomass were subjected to water infusion before the homogenization step, with a dry biomass/water ratio of 1:20, at a temperature of 25° C. (cold extraction of hydrophilic molecules). The infusion occurred in reactors under stirring. After the infusion, the liquid was separated by centrifugation, at a temperature not higher than 45° C., concentrated by direct osmosis and rejoined to the hydrolyzed biomass in the process final steps, at a temperature lower than 45° C. The solid biomass, coming from centrifugation is re-dispersed in water at a ratio dry substance (DS): water of 1:25 and intended to the subsequent homogenization step.

10 kg of dried powder of moringa (Control) were suspended in water with a dry biomass/water ratio of 1:25 and subjected to three high-pressure homogenization cycles at pressure of, respectively, I. 200 bar (20000 kPa), II. 400 bar (40000 kPa), and III. 600 bar (60000 kPa). The homogenized biomass was subject to enzymatic hydrolysis cycles wherein in each cycle one enzyme CELLUSOFT SUPREME 0.5%, pH 8.5, 45° C., ALCALASE 0.5%, pH 7.0, 60° C., PROLYVE NP Liquide 0.6%, pH 6.8, 45° C. After performing enzymatic inactivation, the hydrolysate of non-animal origin was recovered.

The amount of thermosensitive water-soluble molecules in the extract obtained subjecting the biomass to infusion before homogenization and hydrolysis (according to the invention) and in the hydrolysate obtained from the biomass directly subjected to homogenization and hydrolysis (Control) were determined via Mass spectrometry. The results are reported in Table 3 below.

TABLE 3

| | | extract (without | extract (with |
|---|---|---|---|
| Parameter | U.M. | infusion) Control | infusion) |
| Dry matter | g | 2709.16 | 3580.58 |
| Total nitrogen | g | 80.64 | 100.34 |
| Total carbon | g | 736.02 | 1296.34 |
| Total aminoacids | g | 344.23 | 533.81 |
| Total phytohormones (mg) | mg | 709.62 | 2750.94 |
| of which | | | |
| Tryptamine | mg | 0.82 | 0.974 |
| Tryptophan | mg | 587.97 | 2562.89 |
| cis-zeatin | mg | 0.75 | 1.04 |
| N6-isopentenyl-adenine | mg | 0.70 | 0.92 |
| IAA*-Asp | mg | 74.73 | 127.87 |
| IAA*-Glu | mg | 1.83 | 2.85 |
| 2-metylthio-cis-zeatin | mg | 0.28 | 0.84 |
| N6-isopentenyl-adenosine | mg | 0.14 | 0.89 |
| Salycilic acid | mg | 15.18 | 12.89 |
| Acido Jasmonico (JA) | mg | 1.11 | 13.47 |
| IAA*-Phe | mg | 0.02 | 0.16 |
| 2-metylthio- | mg | 0.07 | 3.62 |
| isopentenyladenine | | | |
| Brassinolide | mg | 1.52 | 3.75 |

*indolacetic acid

As it stems from the results of Table 3, such infusion allowed to extract thermosensitive water-soluble molecules, preserving them from degradation due to the higher temperatures used in the subsequent homogenization and/or hydrolysis steps.

The infusion step results in a final extract that contains almost four times more hormones than the Control, confirming that this operation preserves otherwise heat-sensitive molecules that would be partially degraded during the hydrolysis phase.

1. A process to obtain hydrolyzates of non-animal origin comprising the steps of:
i) subjecting a biomass to water infusion with a dry biomass/water ratio comprised between 1:5 and 1:20, at a temperature between 20° C. and 45° C., separating a liquid phase from the solid biomass;
ii) subjecting the solid biomass obtained after step i) of non-animal origin to two or more high-pressure homogenization cycles wherein the homogenization cycles are performed at pressures from 200 bar (20000 kPa) to 2000 bar (200000 kPa);
iii) subjecting the thus obtained homogenized to one or more enzymatic hydrolysis cycles wherein in each cycle one enzyme or more enzymes are used, selected from polysaccharide-degrading enzymes, endo-proteolytic enzymes, or exo-proteolytic enzymes;
iv) performing enzymatic inactivation; and
v) recovering the hydrolyzate of non-animal origin and rejoining with the liquid phase obtained in step i).

2. The process according to claim 1, wherein the biomass of non-animal origin is consisting of plants or plant parts, seed flours, microalgae, or yeasts.

3. The process according to claim 1, wherein, before the homogenization step, the biomass is subjected to cold extraction of lipophilic molecules and the lipophilic extract is separated.

4. The process according to claim 1, wherein the high-pressure homogenization cycles are preferably two, three or four and are performed at the same pressure or at increasing pressures.

5. The process according to claim 1, wherein the polysaccharide-degrading enzymes are selected from cellulases, hemicellulases, glycosidases, pectinases, chitinases, and chitosanases, and mixtures thereof.

6. The process according to claim 1, wherein the endo-proteolytic enzymes are selected from alcalases, serin proteases.

7. The process according to claim 1, wherein the enzymatic hydrolysis comprises the following steps:
a) possible pH adjustment between 4.5 and 6.0, preferably 5.5;
b) possible adding of an enzymatic inoculum of polysaccharide-degrading enzymes e.g., cellulases and/or hemicellulases and/or glycosidases and/or pectinases and/or chitinases and/or chitosanases etc., and execution of an enzymatic infusion for degrading polysaccharides at a temperature comprised between 36 and 73° C., preferably 60° C.;
c) pH adjustment between 6.0 and 8.0, preferably between 6.0 and 7.0 or between 7.0 and 8.0, more preferably between 6.5 or 7.4;
d) adding of at least one endo-proteolytic enzyme ed execution of an endo-proteolytic enzymatic infusion at a temperature comprised between 4° and 50° C., preferably 45° C. or at a temperature comprised between 6° and 75° C., preferably 68° C.;
e) pH adjustment between 6.0 and 7.0, preferably 6.5;
f) adding of at least one exo-proteolytic enzyme ed execution of an exo-proteolytic enzymatic infusion at a temperature comprised between 4° and 50° C., preferably 45° C.;
g) centrifugation and/or filtration to separate the liquid phase from the solid phase;
h) concentration of the liquid phase, preferably by nanofiltration and reverse osmosis; or
under vacuum concentration; or direct osmosis; or combinations thereof.

8. The process according to claim 1, wherein the enzymatic inactivation occurs by heating at a temperature between 3° and 50° C. and/or acidification at a pH between 3.8 and 5.0, and/or subjecting the mixture at a pressure between 20 mbar (2000 Pa) and 200 mbar (20000 Pa) for a period of time between 15 and 30 minutes.

9. The process according to claim 1, wherein before the enzymatic inactivation or after the enzymatic inactivation, the liquid phase is separated from the solid and concentrated.

10. The process according to claim 1, wherein the recovery of the hydrolyzate of non-animal origin occurs by decantation and subsequent filtration and/or centrifugation.

11. Hydrolyzate of non-animal origin obtainable by the process of claim 1.

12. Use of an hydrolyzate of non-animal origin according to claim 11 as a plant biostimulator.
